(19) United States
(12) Patent Application Publication
(10) Pub. No.: US 2006/0079851 A1
(43) Pub. Date: Apr. 13, 2006

(54) Multipurpose eye dropper and fountain device

(76) Inventor: Richard P. Guerrieri, Naples, FL (US)

Correspondence Address: Mark L. Weber, Esq.;Renner, Kenner, Greive, Bobak, Taylor & Weber, Fourth Floor, First National Tower, Akron, OH, 44308-1456, US

(21) Appl. No.: 10/962,151

(22) Filed: Oct. 7, 2004

(51) Int. Cl. A61M 35/00

(52) U.S. Cl. 604/295

(57) ABSTRACT

An eye dropper and fountain device includes a flexible bulb having a hollow interior. A base one-way valve communicates with the hollow interior of the flexible bulb and allows for the flow of air therethrough into the hollow interior. A top one-way valve communicates with the hollow interior of the flexible bulb and allows for the flow of fluid therethrough out of the hollow interior. Solution delivery may be in the form of drops or streams or fountains.

### TECHNICAL FIELD

The present invention generally resides in the art of eye droppers and, more particularly, resides in the art of multipurpose eye dropper and fountain devices wherein a useful eye treatment solution may be delivered to a user's eye in a plurality of different ways selected by the user.

### BACKGROUND ART

Eye droppers and eye fountains have been provided in the prior art. Generally, eye droppers include a nozzle having an open end that communicates with a flexible bulb. The flexible bulb contains an eye treatment solution such that the eye dropper may be upended to point the open end downward and feed the solution into the nozzle, by gravity. In this orientation, slight pressure applied to the flexible bulb forces a drop of the eye treatment solution into the user's eye. In these well known configurations, the user must throw back his head to introduce the eye treatment solution to his eye.

Eye fountains are somewhat similar in that they typically contain a nozzle with an open end, as well as a flexible bulb containing eye treatment solution; however, an eye cup generally surrounds the nozzle such that the eye treatment solution can be fed to the cup in an upright position. Once the cup contains eye treatment solution, air can be introduced through the opened end of the nozzle, while maintaining the eye fountain in an upright position, and the air will bubble through and agitate the eye treatment solution retained in the eye cup. When the user's eye is placed over the eye cup, the agitation will cause the eye treatment solution to contact the user's eye.

Popular and well known eye droppers are supplied under such brand names as Visine™ and Clear Eyes™. Also, various contact lens saline solution eye droppers are well known. Eye droppers are sometimes provided with eye cups, such as in U.S. Pat. No. 3,945,381, to prevent the user from poking his eye with the nozzle tip during an inverted eye solution delivery. Eye fountains are less well known, being suited mainly for specialized applications, such as in a chemical lab. Prior art eye fountains include U.S. Pat. Nos. 1,900,201; 2,524,720; 2,818,068; and 3,261,355.

### SUMMARY OF THE INVENTION

Without limitation, the present invention provides an eye dropper and fountain device that includes a flexible bulb having a hollow interior. A base one-way valve communicates with the hollow interior of the flexible bulb and allows for the flow of air therethrough into the hollow interior. A top one-way valve communicates with the hollow interior of the flexible bulb and allows for the flow of fluid therethrough out of the hollow interior.

In particular embodiments, an eye cup is provided for fitting over a user's eye, and the top one-way valve communicates with the eye cup to allow for the flow of fluid into the eye cup from the hollow interior. In some embodiments, a base cap selectively engages the base one-way valve to selectively open and close the base one-way valve and, by covering the same, serves to keep it sterile.

Various one-way valve configurations are disclosed, as are means for achieving different types of solution delivery, as, for example, streams, mist, drops, and fountain solution delivery.

### BRIEF DESCRIPTION OF THE DRAWINGS

For a complete understanding of the objects, techniques and structure of the invention reference should be made to the following detailed description and accompanying drawings wherein:

FIG. 1 is an assembly diagram of the multi-purpose eye dropper and fountain device of this invention;

FIG. 2 is a general schematic cross-sectional assembly view;

FIG. 3 shows the elements of FIG. 2, as assembled, and in cross section;

FIG. 4 shows the functioning of the multipurpose eye dropper and fountain device during the compression of the flexible bulb;

FIG. 5 shows the functioning of the multipurpose eye dropper and fountain device during the decompression of the flexible bulb;

FIG. 6 is a cross sectional view of a particular embodiment of a one-way valve that may be used in the multipurpose eye dropper and fountain device, shown in a closed position;

FIG. 7 shows the valve of FIG. 6 in an open position;

FIG. 8 is a perspective view of the valve body portion of the one-way valve of FIG. 6, wherein the body portion has been cut in half to show the cavity and channels formed therein for receipt of a valve piston;

FIG. 9 is a side view of a valve piston for use in the one-way valve of FIG. 6;

FIG. 10 is a top view of a valve piston for use in the one-way valve of FIG. 6;

FIG. 11 is a cross sectional view of an alternative embodiment of a one-way valve, wherein the valve piston is stationary, having flexible valve flaps;

FIG. 12 is a cross sectional view of an eye cup, showing how the eye cup may connect to a top one-way valve;

FIG. 13 is a cross sectional view of another alternative embodiment of a top one-way valve according to this invention, wherein the one-way valve, together with a particularly preferred eye cup, provides for the production of different solution delivery streams at the valve outlets;

FIG. 14 is a top view of the valve of FIG. 13;

FIG. 15 is a top view of an eye cup that is employed with the one-way valve of FIGS. 13;

FIG. 16 is an exploded view of the interaction of the valve of FIG. 13 and the eye cup of FIG. 15, with the eye cup in a “mist” position;

FIG. 17 is an exploded view of the interaction of the valve of FIG. 13 and the eye cup of FIG. 15, with the eye cup in a “droplet” position;

FIG. 18 is an exploded view of the interaction of the valve of FIG. 13 and the eye cup of FIG. 15, with the eye cup in a “stream” position;

FIG. 19 is a top view of an alternative embodiment of an eye cup that works in conjunction with a stream selection disk to provide different types of solution delivery streams at the outlet of the top of the one-way valve;

FIG. 20 is a top view of a stream selection disk;

FIG. 21 is a side view of the disk of FIG. 20.

### PREFERRED EMBODIMENT FOR CARRYING OUT THE INVENTION

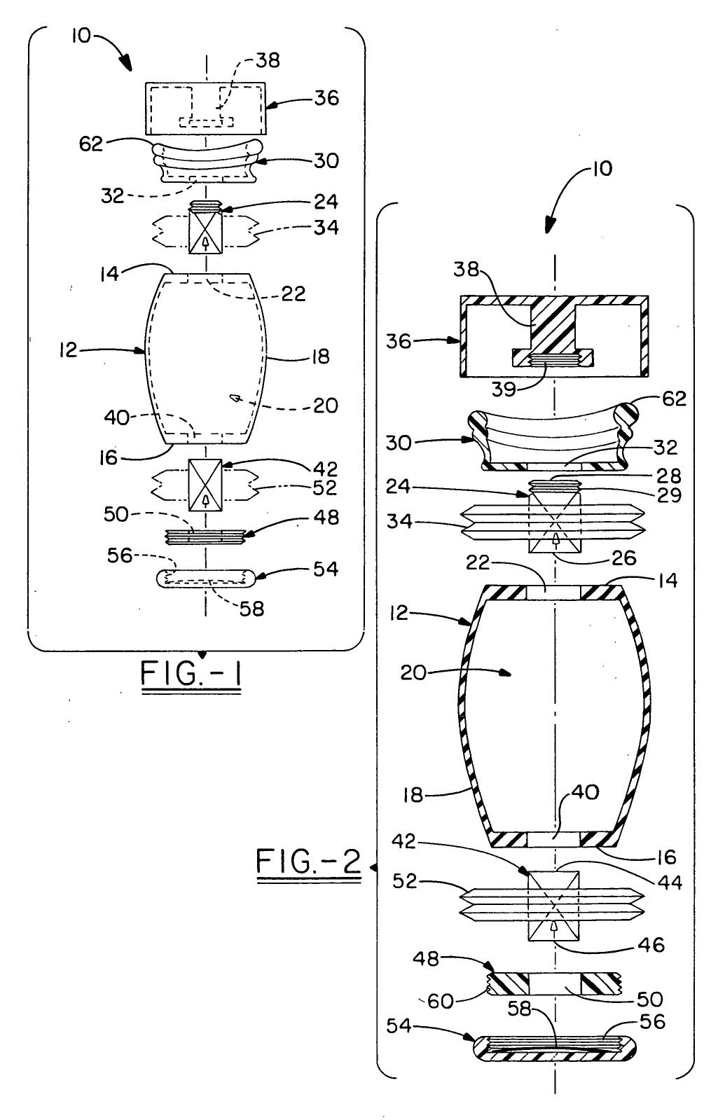

Referring now to FIGS. 1-3, the multipurpose eye dropper and fountain device (herein eye dropper) according to this invention is shown and designated generally by the numeral 10. Eye dropper 10 includes flexible bulb 12 having top 14 and bottom 16 and flexible sidewall 18 defining hollow interior 20, which retains any appropriate desired eye treatment solution.

Top 14 includes aperture 22 which securedly receives top one-way valve 24. More particularly, top one-way valve 24 includes valve inlet 26 that communicates with hollow interior 20 of flexible bulb 12, with valve outlet 28 opposed to valve inlet 26. Eye cup 30 fits over top one-way valve 24, particularly with aperture 32 being securedly affixed around valve outlet 28. In the figures, the general bellows-type configuration of the sidewall 34 of top one-way valve 24 is merely for aesthetics.

Top cap 36 selectively covers eye cup 30, fitting on eye dropper 10 through a selectable engagement of plug member 38 and valve outlet 28. Plug member 38 extends from top cap 36 to cover valve outlet 28, preventing valve outlet 28 from becoming contaminated and preventing eye treatment solution from leaving flexible bulb 12 via outlet 28. In an alternative embodiment, plug member 38 includes a threaded recess 39 that communicates with threads 29 on outlet 28. If desired, top cap 36 may be designed to be removed from eye cup 30 and snapped onto the base of the eye dropper 10 with a common snap fit (or may otherwise be designed to remain associated with the eye dropper 10 when removed from covering the eye cup 30.

Bottom 16 of flexible bulb 12 includes aperture 40, which securedly receives base one-way valve 42. More particularly, base one-way valve 42 includes valve outlet 44, which communicates with hollow interior 20 of flexible bulb 12 through aperture 40. Valve inlet 46 is opposed to valve outlet 44. Base member 48 fits over valve inlet 46, at aperture 50. The general bellows-type configuration of the sidewall 52 of base one-way valve 42 is merely for aesthetics.

Base cap 54 selectively covers base member 48, receiving base member 48 in recess 56. Preferably, base cap 54 mates with base member 48 through a threaded engagement, although other means of engagement might be employed. When base cap 54 is completely threaded over base member 48, seal surface 58 of base cap 54 lies flush with valve inlet 46. By turning base cap 54 relative to base member 48, seal surface 58 is removed from contact with valve inlet 46 according to the pitch of the mating threads, represented at 60. Seal surface 58 may be a resilient material that is bowed upwardly to provide a very effective seal at valve inlet 46.

Top one-way valve 24 allows for the flow of fluid (gas and/or liquid) only in the direction of arrow A, from hollow interior 20 to and out of valve outlet 28. This advantageously prohibits the contamination of the fluid in bulb 12. Base one-way valve 42, which is opposite top one-way valve 24 (at the bottom of flexible bulb 12), allows for the flow of fluid (typically air) only in the direction of arrow B, from outside valve inlet 46, into hollow interior 20.

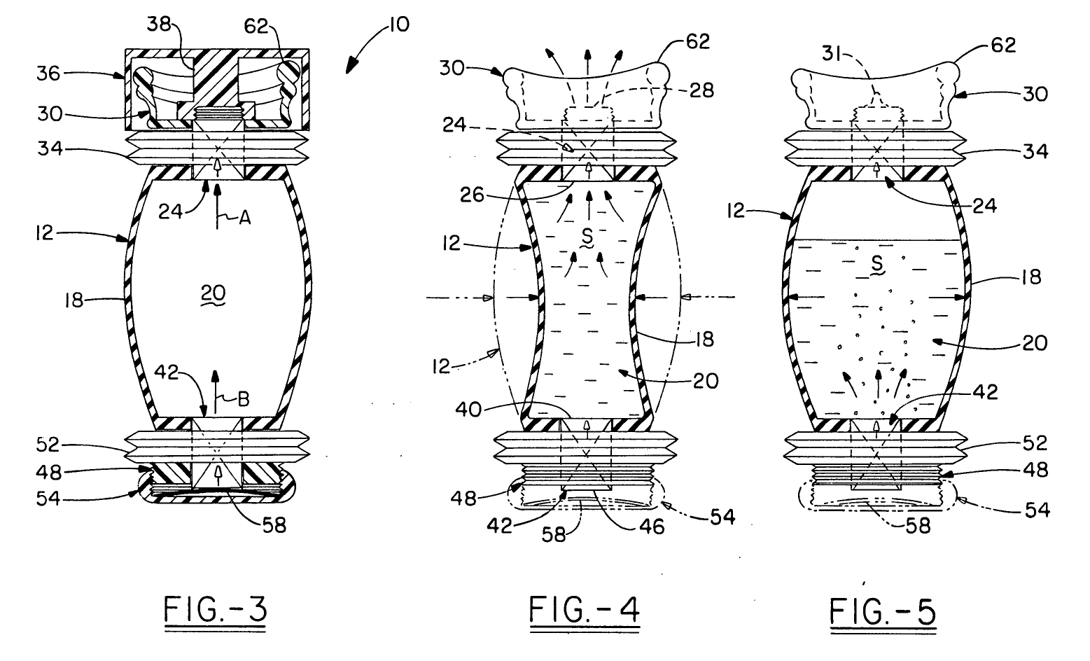

Referring now to FIG. 4, it can be seen that flexible bulb 12, will be at least partially filled by a desired eye treatment solution, which is designated generally by the letter S. Preferably, 80 to 95% of the volume of bulb 12 is filled with such solution. By applying pressure to flexible sidewall 18 of bulb 12 (i.e., by squeezing bulb 12), solution S is advanced to contact valve inlet 26, and, if sufficient pressure is applied, is advanced into top one-way valve 24, and out of valve outlet 28. If eye dropper 10 is upright, as in FIG. 4, squeezing bulb 12 will first force the air above the solution to be advanced through valve 24, until the solution contacts and is then forced through valve 24. In an inverted position, the solution will already be in contact with valve 24, and squeezing bulb 12 will cause the solution to be forced through valve 24. While bulb 12 is squeezed, no solution S exits at base one-way valve 42, because base one-way valve 42, as mentioned, only allows for the flow of fluid (typically air) in the direction of arrow B.

With eye dropper 10 inverted over an eye, squeezing bulb 12 will allow a user to introduce solution S to his eye. With light pressure, a droplet will form at valve outlet 28, and drop to the eye as is generally known. A droplet tip 31 may optionally be provided as an outlet, as shown in phantom. Also, with more pressure, a stream of solution may be forced out at valve outlet 28 or droplet tip 31.

After squeezing bulb 12, it will remain deformed, as top one-way valve 24 does not allow for the flow of fluid into hollow interior 20, and valve inlet 46 of base one-way valve 42 is essentially plugged by contact with seal surface 58. As seen in FIG. 5., to return bulb 12 back to its non-compressed state, base cap 54 is manipulated to distance seal surface 58 from valve inlet 46, allowing outside air to flow into hollow interior 20. Bulb 12 is preferably constructed to favor its non-deformed shape, such that, once valve inlet 46 is no longer stopped by seal surface 58 of base cap 54, air will be drawn in through the tolerances in threads 60 between base member 48 and base cap 54.

It should be appreciated that base cap 54 and its seal surface 58 are optional elements, because base one-way valve 42 does not necessarily have to be sealed by contact with a seal surface. But base cap 54 is preferred because it prevents valve inlet 46 from being contaminated, for example, through contact with dirt or dust.

This ability to refill the remainder of hollow interior 20 with air from the outside allows eye dropper 10 to optionally function as an eye fountain. On method of such use involves holding eye dropper 10 either upright or slightly angled downwardly, squeezing bulb 12 sufficiently to force some of solution S through top one-way valve 24 and into eye cup 30, with enough solution S being introduced to at least cover valve outlet 28 (FIG. 4) when eye dropper 10 is positioned upright. Then, base cap 54 is manipulated to allow air back into hollow interior 20, through base one-way valve 42, as disclosed above (FIG. 5). At this point, with eye dropper 10 in a generally upright position, the user may place his eye over eye cup 30, to rest against bumper 62, and squeezing bulb 12 further, now that it is expanded with air, will cause air to flow through top one-way valve 24 and agitate the solution now in eye cup 30. The agitation will cause a fountain of solution to bathe the user's eye. It is also possible to provide a fountain effect by initially flushing the eye by sealing bumper 62 around an eye in an inverted position and squeezing bulb 12 to provide an initial flush; thereafter moving eye dropper 10 upright and, with solution in eye cup 30 from the initial flush, expanding bulb 12 and squeezing to bubble air through the solution.

One-way valves 24 (top) and 42 (base) may take any form suitable for serving their intended functions of allowing fluid travel there through in one direction, as disclosed. However, certain embodiments, particularly for top one-way valve 24 are envisioned. Although these alternative embodiments are particularly preferred for use as top one-way valves 24, it will be appreciated they might also be employed as base one-way valves 42 or both.

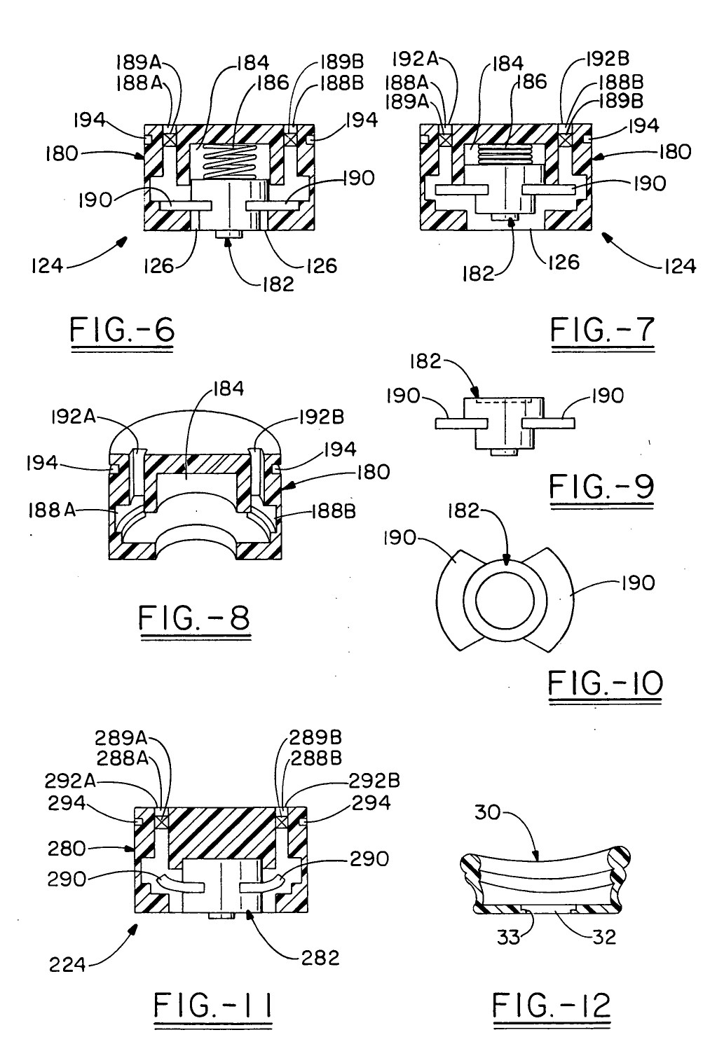

An embodiment of a one-way valve is shown in FIGS. 6-10 and designated by the numeral 124. Valve 124 includes valve body 180 and valve piston 182. As seen in cross section in FIGS. 6 and 7, piston 182 moves within cavity 184 of valve body 180, against a bias means 186. More particularly, valve body 180 has solution delivery channels 188A and 188B, and piston 182 moves against bias means 186 to block or unblock the communication between channels 188A, 188B and the valve inlet 126, which communicates with the hollow interior of bulb 12, when valve 124 is affixed thereto, as already disclosed with respect to the more generic valve 24. More particularly, by squeezing flexible bulb 12, the fluid in bulb 12 will push against flaps 190, which block communication between channels 188A, 188B and the interior of bulb 12, and the force against flaps 190 will move valve piston 182 to overcome bias means 186, opening up the communication between channels 188A, 188B and the interior of flexible bulb 12. This is seen in the closed position of FIG. 6 and the open position of FIG. 7. Thus, solution may be delivered to the valve outlets 192A, 192B provided by channels 188A, 188B. The bias means may be any suitable material or device that urges valve piston 182 to the position shown in FIG. 6, and yet move upwardly against pressure created upon squeezing bulb 12. As non-limiting examples, a spring or resilient elastomer material may be employed.

In FIG. 11, an alternative embodiment of a one-way valve is shown designated by the numeral 224. Valve 224 includes body portion 280 and piston 282, although piston 282 is not a true “piston” in that it does not move within body portion 280. Rather, piston 282 is fixed to body portion 280, and may even be formed integrally therewith, and it is the flaps 290 that flex to allow solution to travel from within flexible bulb 12 through channels 288A, 288B and outlets 292A, 292B. In this embodiment, flaps 290 are appropriately flexible whereas the flaps 190 are rigid in the movable piston embodiment of FIGS. 6-10.

In the embodiments of FIGS. 6, 7 and 11, channels 188A, 188B, 288A and 288B may be provided with pinch valves 189A, 189B, 289A and 289B, respectively. These pinch valves substantially seal the outlet end of the channels until solution is forced past them at sufficient pressure through squeezing of bulb 12. This is an added benefit, because, even when there is very little solution left in bulb 12, it is possible to invert eye dropper 10, fill the solution delivery channels, allow the piston to return to its bias position to trap the solution in the channels, position the eye dropper upright, and then create a minor fountain effect by subsequent squeezing of the bulb to force air and solution through the pinch valves.

With reference to FIG. 12, the valve embodiments shown in FIGS. 6-11 include circumferential detents 194 (FIG. 6 embodiment) and 294 (FIG. 11 embodiment) that communicate with circumferential protrusion 33 at aperture 32 of eye cup 30. In preferred embodiments, the materials forming eye cup 30 and valve 124 or 224 are sufficiently resilient such that the circumferential protrusion 33 can fit into the circumferential detent 194, 294 through a “snap” fit, although other means of securing may be employed.

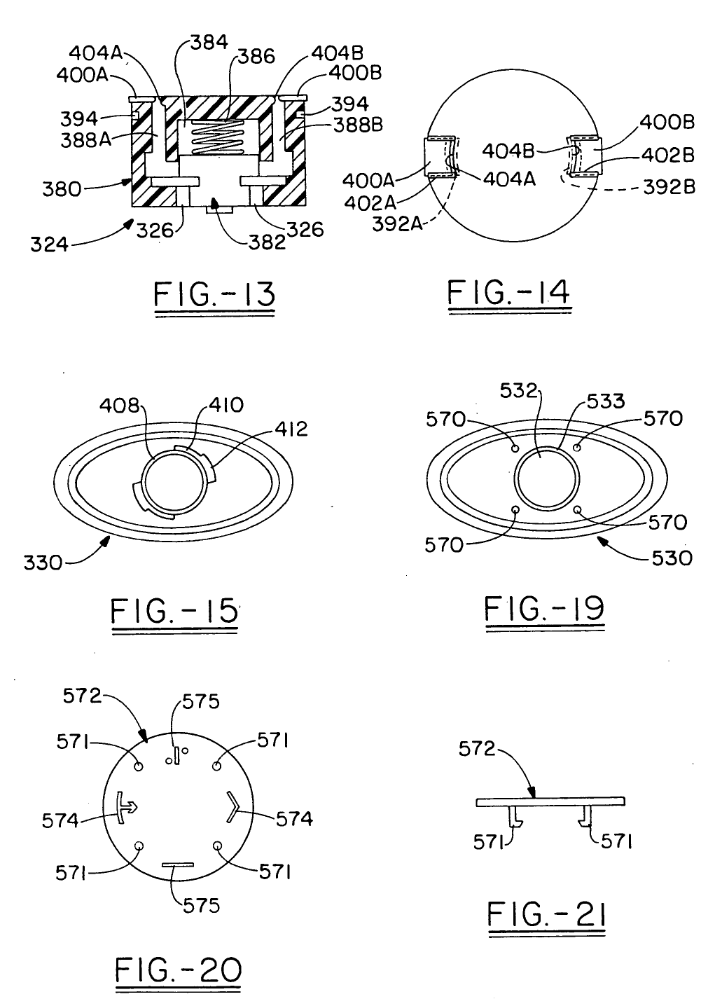

Referring now to FIGS. 13-18, a particularly preferred embodiment for a valve is shown and designated by the numeral 324, and a preferred eye cup for interaction with valve 324 is shown and designated by the numeral 330. Valve 324 includes body portion 380 and piston 382. In the embodiment shown, piston 382 is biased by bias means 386, although piston 382 may be changed to function as piston 282 above. More particularly, valve body 380 has solution delivery channels 388A and 388B, and piston 382 moves against bias means 386 to block or unblock the communication between channels 388A, 388B and valve inlet 326, which communicates with the hollow interior of bulb 12. Valve 324 differs from other disclosed valves by having resistor tabs 400A, 400B retained within grooves 402A, 402B and capable of moving therein. More particularly, resistor tabs 400A, 400B are retained within their respective groove 402A, 402B, adjacent solution delivery channels 388A, 388B, and, in the position shown in FIG. 14, abut a channel protrusion 404A, 404B, respectively, to substantially block outlets 392A, 392B.

Referring now to FIG. 15, showing a top view of eye cup 330, grooves 402A, 402B align with circumferential steps 408, 410 and 412. More particularly, as shown in the exploded views of FIGS. 16-18, eye cup 330 is removably secured to valve 324 at circumferential detent 394 (on valve 334) and circumferential protrusion 333 (substantially like protrusion 33 of eye cup 30, FIG. 12). Circumferential steps 408, 410, and 412, as seen in FIGS. 16-18, are formed in eye cup 330, above, protrusion 330, and provide an area where resistor tabs 400A, 400B may move into as solution is being forced through channels 388A, 388B, and out of valve outlets 392A, 392B. Eye cup 330 may be rotated around valve 324, through the interaction of detent 394 and protrusion 333. As eye cup 330 is rotated, resistor tabs 400A, 400B are selectively aligned with one of the circumferential steps 408, 410, 412, and solution forced through channels 388A, 388B push against resistor tabs 400A, 400B to cause them to move away from channel protrusions 404A and 404B, to a differing degree, to cause different types of solution delivery.

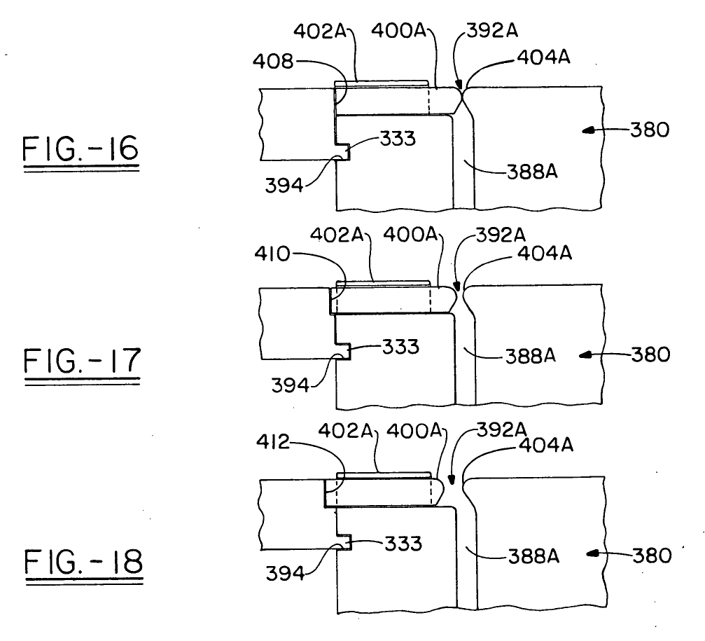

In FIG. 16, resistor tab 400A is shown in alignment with circumferential step 408. In this position, resistor tab 400A (as well as resistor tab 400B, not shown) abuts against circumferential step 408, and cannot move to the left (in the orientation shown in the figure), such that the solution is delivered at outlet 392A as a mist, being forced through two abutting surfaces. In FIG. 17, resistor tab 400A aligned with circumferential step 410 such that the resistor tab is forced away from channel protrusion 404A as solution is forced through channels 388A, 388B, and solution is thus delivered at outlet 392A as a droplet. In FIG. 18, resistor tab 400A is aligned with circumferential step 412 such that solution forced through channel 388A forces resistor tab 400A even further away from channel protrusion 404A, allowing the solution to be delivered at outlet 392A as a stream. It will be appreciated that, in all instances, the same holds true for resistor tab 400B, channel 388B, channel protrusion 404B, and valve outlet 392B. Thus, in this particularly preferred embodiment, by rotating eye cup 330, different types of solution delivery may be achieved.

Different types of solution delivery may also be achieved through yet another embodiment of an eye cup, which is designated in FIG. 19 as eye cup 530. Eye cup 530 includes aperture 532 and circumferential protrusion 533, and can be fit to valves in accordance with the disclosure above. Bores 570, receive legs 571 of disk 572, as shown in FIGS. 20 and 21, so that disk 572 fits over the valve outlets of the valve. More particularly, disk 572 includes two pairs of solution apertures, a first pair disposed horizontally in the figure and designated by the numeral 574, and a second pair disposed vertically in the figure and designated by the numeral 575. The solution apertures 574, 575 are selectively aligned with valve outlets (disclosed above) upon rotating eye cup 530 about the valve to which it is affixed, substantially as disclosed with respect to the eye cup embodiment of FIGS. 15-18 (although this embodiment does not include circumferential steps). Different shapes are provided for the solution apertures on disk 572 in order to show that the solution apertures may be shaped in special ways to produce special solution delivery.

Thus it can be seen that the objects of the invention have been satisfied by the structure presented above. While in accordance with the patent statutes only the best mode and preferred embodiment of the invention has been presented and described in detail, the invention is not limited thereto or thereby. Accordingly, for an appreciation of the scope and breadth of the invention reference should be made to the following claims.

1. An eye dropper and fountain device comprising:
a flexible bulb having a hollow interior;
a base one-way valve communicating with said hollow interior of said flexible bulb, said base one-way valve allowing for the flow of fluid therethrough into said hollow interior; and
a top one-way valve communicating with said hollow interior of said flexible bulb, said top one-way valve allowing for the flow of fluid therethrough out of said hollow interior.

2. The eye dropper of claim 1, further comprising an eye cup for fitting over an eye, said top one-way valve communicating with said eye cup to allow for the flow of fluid into said eye cup from said hollow interior.

3. The eye dropper of claim 1, further comprising a base cap selectively engaging said base one-way valve to selectively open and close said base one-way valve and limit its contact with outside elements to keep it sterile.

4. The eye dropper of claim 1, where said top one-way valve comprises:
at least one solution delivery channel having an inlet communicating with said hollow interior of said bulb and an outlet communicating with the exterior of the eye dropper.

5. The eye dropper of claim 4, further comprising a piston biased by bias means to close the communication of said at least one solution delivery channel between said interior and exterior, said piston being selectively moved against said bias means by applying pressure thereto through squeezing of said bulb, with such movement serving to open the communication of said at least one solution delivery channel between said interior and exterior.

6. The eye dropper of claim 5, wherein said bias means is selected from the group consisting of resilient materials and springs.

7. The eye dropper of claim 4, further comprising:
an eye cup for fitting over an eye, said top one-way valve communicating with said eye cup to allow for the flow of fluid into said eye cup from said hollow interior;
a resistor tab retained within a groove positioned proximate the outlet of said at least one solution delivery channel, said resistor tab selectively moving within said groove to selectively open and close the communication of said at least one solution delivery channel between said interior and exterior.

8. The eye dropper of claim 7, wherein said eye cup is secured to the eye dropper to rotate about said top one-way valve, said eye cup providing a plurality of circumferential steps that selectively align with said resistor tab to selectively allow for greater or lesser movement of said resistor tab within said groove to selectively open and close the communication of said at least one solution delivery channel between said interior and exterior to a greater or lesser degree.

9. The eye dropper of claim 1, where said top one-way valve comprises:
at least one solution delivery channel communicating between said hollow interior of said bulb and the exterior of the eye dropper;
a stopper having at least one resilient flap that closes the communication of said at least one solution delivery channel between said interior and exterior, said flap being selectively moved to open the communication by applying pressure thereto through squeezing of said bulb.

10. The eye dropper of claim 2, further comprising an eye cup for fitting over an eye, said top one-way valve communicating with said eye cup to allow for the flow of fluid into said eye cup from said hollow interior, wherein said eye cup is secured to the eye dropper to rotate about said top one-way valve, said eye cup providing a plurality of solution apertures that, upon rotation of said eye cup selectively align with said at least one solution delivery channel, said plurality of solution apertures providing different types of solution delivery.

11. An eye dropper and fountain device comprising:
a flexible bulb having a hollow interior;
a top one-way valve having at least one solution delivery channel with an inlet communicating with said hollow interior of said bulb and an outlet communicating with the exterior of the eye dropper, said top one-way valve allowing for the flow of fluid through said at least one solution delivery channel out of said hollow interior;
an eye cup for fitting over an eye, said at least one solution delivery channel of said top one-way valve communicating with said eye cup to allow for the flow of fluid into said eye cup from said hollow interior; and
a resistor tab retained within a groove positioned proximate the outlet of said at least one solution delivery channel, said resistor tab selectively moving within said groove to selectively open and close the communication of said at least one solution delivery channel between said interior and exterior.

12. The eye dropper of claim 11, wherein said eye cup provides a plurality of circumferential steps that selectively align with said resistor tab to selectively allow for greater or lesser movement of said resistor tab within said groove to selectively open and close the communication of said at least one solution delivery channel between said interior and exterior to a greater or lesser degree.

13. The eye dropper of claim 12, further comprising a base one-way valve communicating with said hollow interior of said flexible bulb, said base one-way valve allowing for the flow of fluid therethrough into said hollow interior of said flexible bulb.

14. An eye dropper and fountain device comprising:
a flexible bulb having a hollow interior; and
a top one-way valve communicating with said hollow interior of said flexible bulb and allowing for the flow of fluid therethrough out of said hollow interior, said top on-way valve including:
at least one solution delivery channel having an inlet communicating with said hollow interior of said bulb and an outlet communicating with the exterior of the eye dropper,
a piston biased by bias means to close the communication of said at least one solution delivery channel between said interior and exterior, said piston being selectively moved against said bias means by applying pressure thereto through squeezing of said bulb, with such movement serving to open the communication of said at least one solution delivery channel between said interior and exterior, and
a pinch valve substantially sealing said outlet of said at least one solution delivery channel but allowing solution to be forced through the seal provided by said pinch valve when sufficient pressure is applied to said flexible bulb.

15. The eye dropper of claim 14, further comprising a base one-way valve communicating with said hollow interior of said flexible bulb, said base one-way valve allowing for the flow of fluid therethrough into said hollow interior.
